(19) United States
(12) Patent Application Publication
(10) Pub. No.: US 2016/0052887 A1
(43) Pub. Date: Feb. 25, 2016

(54) PHARMACEUTICALLY ACCEPTABLE AMINE SALTS OF PITAVASTATIN

(71) Applicant: DSM SINOCHEM PHARMACEUTICALS NETHERLANDS B.V., Delft (NL)

(72) Inventor: Ben DE LANGE, Echt (NL)

(73) Assignee: DSM Sinochem Pharmaceuticals Netherlands B.V., Delft (NL)

(21) Appl. No.: 14/780,898

(22) PCT Filed: Mar. 28, 2014
(86) PCT No.: PCT/EP2014/056243

§ 371 Date: Sep. 28, 2015

(30) Foreign Application Priority Data
Mar. 29, 2013 (EP) 13161842.3
Aug. 29, 2013 (EP) 13182232.2

(51) Int. Cl. C07D 215/14; C07C 215/08; C07C 215/10; C07C 213/08

(52) U.S. Cl. CPC C07D 215/14; C07C 213/08; C07C 215/08; C07C 215/10

(57) ABSTRACT

The present invention relates to pharmaceutically acceptable amine salts of pitavastatin and a method for producing pharmaceutically acceptable amine salts of pitavastatin. Also provided are pharmaceutical compositions of these amine salts or solvates thereof, and methods of their use as HMG-CoA reductase inhibitors.

### FIELD OF THE INVENTION

The present invention relates to pharmaceutically acceptable amine salts of pitavastatin and a method for producing pharmaceutically acceptable amine salts of pitavastatin. Also provided are pharmaceutical compositions of these amine salts or solvates thereof, and methods of their use as HMG-CoA reductase inhibitors.

### BACKGROUND OF THE INVENTION

HMG-CoA reductase inhibitors, also known as statins, are widely used drugs prescribed to treat hypercholesterolemia, hyperlipoproteinemia, and atherosclerosis. Examples of HMG-CoA reductase inhibitors are atorvastatin, fluvastatin, lovastatin, mevastatin, pitavastatin, pravastatin, rosuvastatin and simvastatin.

Production of HMG-CoA reductase inhibitors includes (bio)-chemical conversion, chromatography, crystallization extraction, fermentation and the like. Some HMG-CoA reductase inhibitors, like lovastatin, are produced by fermentation using microorganisms of different species identified as species belonging to Aspergillus, Monascus, Nocardia, Amycolatopsis, Mucor or Penicillium genus. Some, like mevastatin, pravastatin and simvastatin, are obtained by treating the fermentation products using the methods of chemical or enzymatic synthesis. Others, like atorvastatin, fluvastatin, pitavastatin and rosuvastatin, are the products of total chemical synthesis.

In EP 742209 short chain (1-3) alkyl amine salts of pitavastatin are disclosed. WO 2007/132482 discloses certain amine salts (arginine, dicyclohexylamine, methyl amine, n-, sec- and tert-butyl amine salts) as intermediates in the preparation of pharmaceutically acceptable salts of pitavastatin. Likewise, CZ 2012-322 discloses L-lysine, tert-butyl amine and 1,1,3,3-tetramethylguanidinium salts of pitavastatin. Furthermore WO 2007/132482 discloses a method of preparation of the said amine salts comprising distillation which brings as disadvantage the enhanced risk of formation of unwanted side products through degrading pathways.

For optical resolution of pitavastatin diastereomers, WO 2010/027060 discloses chiral amine salts of pitavastatin with (S)-α-aminobenzeneacetic acid methyl ester, (R)-β-aminobenzenepropanol, (R)-α-ethylbenzenemethanamide, (S)-β-aminobenzene-propanol and (R)-α-methyl-1-naphthalenemethanamine.

With the objective of finding new polymorphs of pitavastatin, WO 2012/106584 discloses various amines such as diethanol amine, diisopropyl amine, meglumine, phenylethyl amine, piperazine, piperidine and n-propyl amine salts of pitavastatin.

Driven by the pressure to avail medication such as HMG-CoA reductase inhibitors at affordable prices, industry is in constant need for process and product rationalization and optimization. A problem associated with the presently employed drug product form of pitavastatin, namely the pharmaceutically acceptable calcium salt of pitavastatin, is it low solubility in aqueous environment. Generally, this problem results in the need to provide relatively high doses of active pharmaceutical ingredient in therapeutic administration and in addition transfer of the active ingredient into the body's targeted fluids is usually slow making the active ingredient susceptible to unwanted degradation processes. There is thus a need for improved pharmaceutically acceptable salts that have improved solubility, are of high purity and which can be prepared using simple and low cost techniques. None of the prior art documents however mentions the use of amine salts as pharmaceutically acceptable salts but merely presents several amine salts for different purposes. From this perspective, it is an aim of the invention to provide alternative pharmaceutically acceptable amine salts of pitavastatin using simple and non-degrading technology; these alternative pharmaceutically acceptable amine salts can be administered as HMG-CoA reductase inhibitors.

### DETAILED DESCRIPTION OF THE INVENTION

In a first aspect, the invention provides a pharmaceutically acceptable amine salt of pitavastatin and solvates thereof (which comprises pitavastatin, a pharmaceutically acceptable amine and a solvent). It was found that pitavastatin readily forms salts with said amines selected from the group consisting of aminopolyols and tetraalkyl ammonium salts that crystallize once they are formed. It has been found that crystals of the amine salt of pitavastatin of high purity may be obtained from solutions comprising a large number of impurities and undesired pitavastatin analogs. Surprisingly, it was found that these salts display increased dissolution speed in aqueous environment whereas the solubility is significantly higher than that of the known pitavastatin calcium salt.

Suitable pharmaceutically acceptable amines for use in the amine salts or solvates thereof as provided herein are tetraalkyl ammonium salts (preferably carnitine and esters thereof, choline, tetraethyl ammonium, tetramethyl ammonium) and aminopolyols (preferably tris(hydroxymethyl)aminomethane). Tris(hydroxymethyl)amino-methane (also referred to as Tris or tromethamine) is one of the most used buffers in molecular biology and cell culture due to its low toxicity, stability and buffering capacity. Tris(hydroxymethyl)aminomethane is described in the Pharmacopoeia and is used as a counter ion in pharmaceuticals.

Yet another preferred amine is choline. Choline (N,N,N-trimethylethanolammonium cation) is a water soluble essential nutrient. Choline is the precursor molecule for the neurotransmitter acetylcholine, which is involved in many functions including memory and muscle control. Choline must be consumed through the diet for the body to remain healthy. It is used in the synthesis of the constructional components in the body's cell membranes.

In another embodiment, the solvent in the solvate is an alcohol, examples of which are 1-butanol, 2-butanol, tert-butanol, ethanol, 2-ethoxyethanol, ethylene glycol, isopropanol, 2-methoxyethanol, 3-methyl-1-butanol, 1-pentanol and 1-propanol.

The pitavastatin amine salts of the present invention are found to have surprising characteristics that offer unexpected opportunities in medical applications. Compared to the known pitavastatin calcium salt, the pitavastatin amine salts of the present invention display increased dissolution speed in aqueous environment whereas the solubility (in aqueous environment) of the pitavastatin amine salts of the present invention is significantly higher than that of the pitavastatin calcium salt. Preferably the solubility of the pitavastatin amine salts of the present invention is 20 times as high as that of pitavastatin calcium salt at the same pH value, more preferably 30 times as high, most preferably from 40 to 250 times as high. The advantage of higher solubility is that a lower dosage of the compound in question can be used to achieve a similar medical effect. Clearly this is advantageous in terms of potential side effects, costs and treatment protocols.

In yet another embodiment, the molar ratio of pitavastatin versus the amine in an amine salt or a solvate thereof is from about 0.5 to about 10, from 0.5 to about 5, from about 0.5 to about 3, from about 0.5 to about 2, or from about 0.8 to about 1.2, or about 1. In certain embodiments, the molar ratio of pitavastatin versus the solvent in a solvate of an amine salt provided herein is from about 0.1 to about 2, from about 0.2 to about 1, or from about 0.3 to about 0.5, or about 0.1, about 0.2, about 0.3, about 0.4, about 0.5, about 0.6, about 0.7, about 0.8, about 0.9, or about 1.

In a second aspect, the present invention provides a process for the preparation of amine salts of pitavastatin and solvates thereof. The process comprises reacting pitavastatin with an amine in a solvent at a first temperature followed by precipitating the amine salt at a second temperature. Preferably said second temperature is at least 5° C. below said first temperature, more preferably from 5° C. to 100° C. below said first temperature, more preferably from 10° C. to 50° C. below said first temperature.

Suitable pharmaceutically acceptable amines for use in the process of the second aspect are manifold and not limited to those described in the first aspect of the invention. Hence, amines suitable for the process of the present invention are ammonia, amino acids (preferably histidine, lysine, ornithine), tetraalkyl ammonium salts (preferably carnitine and esters thereof, choline, tetraethyl ammonium, tetramethyl ammonium), aminopolyols (preferably tromethamine), purines, guanines, vitamins (preferably vitamins B1, B3, B6 and B11), amino sugars (preferably daunosamine, galactosamine, glucosamine, N-methylglucamine) and ethyl amine derivatives (preferably benzathine, diethyl amine, ethanol amine, ethyl amine, ethylene diamine, 1-(2-hydroxyethyl)-pyrrolidine, piperazine, triethanol amine, triethyl amine). For a review on additional amines, see “Handbook of Pharmaceutical Salts: Properties, Selection, and Use” by Stahl and Wermuth, Wiley-VCH, 2002. A preferred amine is lysine, which is a natural occurring amino acid. L-Lysine is a necessary building block for all protein in the body. L-Lysine plays for example a major role in calcium absorption.

In one embodiment, the pharmaceutical acceptable amine is a diamine. The pharmaceutically acceptable diamine has first and second amino groups, which each are independently a primary, secondary, or tertiary amino group, or quaternary ammonium group. Suitable diamines for use in the diamine salt include benzathine, ethylenediamine and piperazine. The diamine salt of pitavastatin comprises from about 1 to about 3, from about 1.5 to about 2.5, from about 1.75 to about 2.25, or about 2 molar equivalents of pitavastatin for one molar equivalent of the diamine.

Clearly, other preferred amines are those described in the first aspect such as tetraalkyl ammonium salts (preferably choline) and polyols (preferably tromethamine)

Suitable solvents for use in preparing the amine salts of pitavastatin include alcohols (such as 1-butanol, 2-butanol, tert-butanol, ethanol, 2-ethoxyethanol, ethylene glycol, isopropanol, methanol, 2-methoxyethanol, 3-methyl-1-butanol, 1-pentanol and 1-propanol), amides (such as N,N-dimethylacetamide, N,N-dimethylformamide and formamide), carbonates (such as ethylene carbonate and propylene carbonate), carbon sulfide, carboxylic acids (such as acetic acid, trichloroacetic acid and trifluoroacetic acid), esters (such as butyl acetate, ethyl acetate, ethyl formate, isobutyl acetate, isopropyl acetate, methyl acetate and propyl acetate), ethers (such as anisole, bis(2-methoxyethyl)ether, diethyl ether, diisopropyl ether, 1,2-dimethoxyethane, 1,1-dimethoxymethane, 2,2-dimethoxypropane, diphenyl ether and methyl tert-butyl ether), halogenated hydrocarbons (such as carbon tetrachloride, chlorobenzene, chloroform, 1,2-dichloroethane, 1,1-dichloroethene, 1,2-dichloroethene, dichloromethane, trichloroethane, trichloroethene, and trifluoromethylbenzene), heterocycles (such as dioxane, N-methyl pyrrolidone, 2-methyl tetrahydrofuran, pyridine and tetrahydrofuran), hydrocarbons (such as benzene, cumene, cyclohexane, cyclopentane, heptane, hexane(s), isooctane, methylcyclohexane, octane, pentane, petroleum ether, tetralin, toluene and xylene), ketones (such as acetone, butanone, methyl butyl ketone, methyl ethyl ketone, methyl methyl isobutyl ketone and isopropyl ketone), nitriles (such as acetonitrile), nitro compounds (such as nitrobenzene and nitromethane), phosphoramides (such as hexamethylphosphoramide), sulfones (such as sulfolane), sulfoxides (such as dimethyl sulfoxide), water and mixtures thereof.

In one embodiment, the amine salt forming reaction is carried out at a first temperature from about −10 to about 110° C., from about 10 to about 80° C. or from about 20 to about 60° C. In many cases the preferred first temperature is found to be at values close to ambient, i.e. from 15 to 45° C. Given the fragile nature of the target compounds this is quite advantageous in view of reduced formation of unwanted side products, such as degradation products.

In another embodiment, the amine salt forming reaction is performed in the presence of an excess amount of the amine to maximize the yield of the reaction. The molar ratio of the amino group on the amine versus pitavastatin is no less than about 1.01, no less than about 1.05, no less than about 1.1, no less than about 1.2, from about 1.05 to about 10, from about 1.1 to about 5, or from about 1.2 to about 2.5. In one embodiment, the salt forming reaction is performed in a solution, that is, both pitavastatin and the amine are dissolved in the solvent. In certain embodiments, the salt forming reaction is performed as a slurry mixture in the solvent, in which case pitavastatin is not fully dissolved whereas the amine is completely dissolved.

In yet another embodiment, it was found that formation of the amine salt of pitavastatin can be combined in a single process step with the deprotection sequence that is usually required in the synthesis of pitavastatin. During production carboxyl and hydroxyl functions of pitavastatin need to be protected and protective groups are removed at the final stage of the synthesis. Removal of protective groups usually includes an acidic treatment. For example, the process may be performed as follows. A protected derivative of pitavastatin, for example the methyl ester of pitavastatin acetonide, is dissolved or suspended in a suitable solvent, for example acetonitrile. Removal of protecting groups may be carried out by treatment with acid followed by treatment with base, or vice versa. Optionally the organic solvent may be changed by distillation followed by addition of a second solvent, for example ethyl acetate. Preferably the aqueous phase is removed after which the amine of choice is added to the organic phase. Preferably the amount of amine added 1.0 to 2.0 mole-equivalents compared to pitavastatin. The resulting mixture can optionally be concentrated in order to reduce mother liquor losses, if any. The desired amine salt of pitavastatin precipitates or crystallizes and can be isolated following simple techniques known to the skilled artisan, such as centrifugation, decantation, filtration and the like. Preferably the salt thus obtained is washed with the same solvent as used for the crystallization/precipitation process. Optionally the amine salt of the HMG-CoA reductase inhibitor may be re-crystallized, for instance from an alternate solvent such as acetonitrile. It was found that the amines of the present invention not only are suitable for formation of stable and pure salts but simultaneously can function to neutralize acidic conditions, thereby preventing the formation of additional foreign salts.

In still another embodiment, the amine salt formed in the amine forming reaction step may be precipitated out from the reaction solution or slurry mixture using conventional methods, including cooling, chilling, solvent evaporation, addition of an anti-solvent or reverse addition to an anti-solvent. Suitable anti-solvents may be chosen from the same list as outlined above, however with the provision that the solubility of the amine in the anti-solvent is below the solubility in the first solvent. Optionally, the solvent and the anti-solvent in a solvent/anti-solvent pair are at least partially miscible. The precipitating step may be carried out at a temperature from about −50 to about 100° C., from about −30 to about 50° C., or from about −10 to about 30° C. To accelerate the precipitation (crystallization) step, the process may further comprise the step of seeding the reaction solution or mixture, prior to or during the initiation of the precipitation step. The amount of seed crystals added exceeds the saturation amount in the solvent being used so that there are un-dissolved seed crystals present in the reaction solution. The process may also comprise an isolation step, in which the precipitate may be isolated by a conventional method, such as filtration and centrifugation, followed by washing with a solvent and then drying. The amine salt may be precipitated by cooling the reaction solution to or below room temperature, or by solvent evaporation.

Other salt forming methods may also be applicable in the present invention. For example, the amine salt of pitavastatin may be prepared by converting a salt of the acid, e.g., sodium salt or potassium salt, to an amine salt via cation exchange using a cation exchange column. The amine salt of pitavastatin may also be produced by physically grinding solid pitavastatin and the amine together in the absence of a solvent.

In addition to precipitation and crystallization, the solid amine salts provided herein may also be prepared using conventional methods known to those skilled in the art, including spray drying, roller drying, lyophilization, and melt crystallization.

In a third aspect, the invention provides a pharmaceutical composition which comprises an amine salt of pitavastatin, or a pharmaceutically acceptable hydrate or solvate thereof, as an active pharmaceutical ingredient, in combination with one or more pharmaceutically acceptable carriers or excipients. The choice of excipient, to a large extent, depends on factors, such as the particular mode of administration, the effect of the excipient on the solubility and stability of the active ingredient, and the nature of the dosage form.

The pharmaceutical compositions of the present invention may be provided in unit-dosage forms or multiple-dosage forms. Unit-dosage forms refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The amine salts of pitavastatin may be administered alone, or in combination with one or more other compounds, one or more other active ingredients. The pharmaceutical compositions that comprise an amine salt provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126). The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### EXAMPLES

### Example 1

### Preparation of Amine Salts of Pitavastatin

The methyl ester of pitavastatin acetonide (5.6 mmol) was added to acetonitrile (21 mL). The mixture was heated to 35° C. until complete dissolution was obtained. To the solution 0.02 N aqueous HCl (9 mL) was added over a period of 1 h. The mixture was stirred for 12 h, followed by addition of 1 N aqueous NaOH in 15 min until pH=12. After stirring for 1 h, the mixture was concentrated under vacuum to remove acetonitrile. Next, ethyl acetate (30 mL) was added followed by addition of 1 N aqueous HCl until pH=4. The ethyl acetate phase was separated. To the ethyl acetate phase was added over a period of 30 min, 1 equiv. (5.6 mmol) of amine dissolved in ethyl acetate (10 mL). Upon addition, a white precipitate was formed. The resulting slurry was stirred for 1 h, followed by filtration of the amine salt of pitavastatin. The amine salt was washed with ethyl acetate (2×5 mL), dried and re-crystallized from acetonitrile.

### Example 2

### Preparation of 2-((4R,6S)-6-((E)-2-(2-cyclopropyl-4-(4-fluorophenyl)quinolin-3-yl)vinyl)-2,2-dimethyl-1,3-dioxan-4-yl)acetate, methyl ester from 2-cyclopropyl-4-(4-fluorophenyl)quinolin-3-carbaldehyde and 2-((4R,6S)-6-((benzo[d]thiazol-2-ylsulfonyl)methyl)-2,2-dimethyl-1,3-dioxan-4-yl)acetate methyl ester

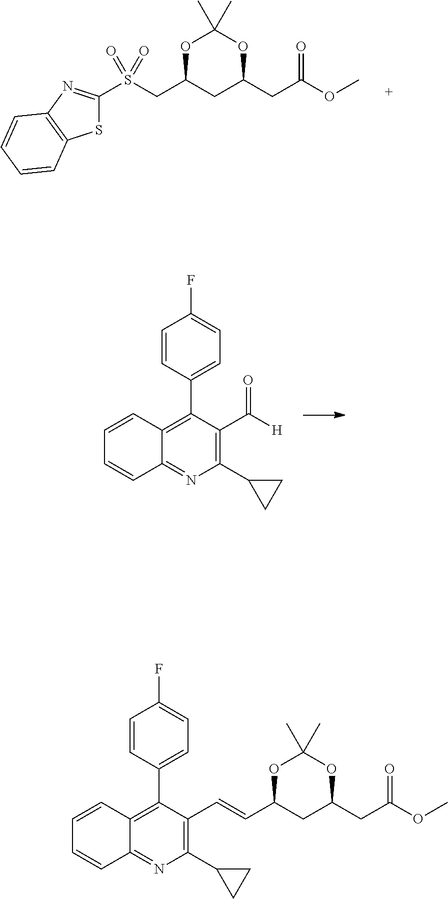 [SMILES: COC(=O)C[C@H]1C[C@@H](CS(=O)(=O)c2nc3ccccc3s2)OC(C)(C)O1 ; O=Cc1c(C2CC2)nc2ccccc2c1-c1ccc(F)cc1 ; COC(=O)C[C@H]1C[C@@H](/C=C/c2c(C3CC3)nc3ccccc3c2-c2ccc(F)cc2)OC(C)(C)O1]

2-((4R,6S)-6-((Benzo[d]thiazol-2-ylsulfonyl)methyl)-2,2-dimethyl-1,3-dioxan-4-yl)acetate methyl ester (35.0 g, 87 mmol) and 2-cyclopropyl-4-(4-fluorophenyl)quinolin-3-carbaldehyde (23.9 g, 82 mmol) were added to 78 mL of N-methyl-2-pyrrolidone and 280 mL of 2-methyltetrahydrofuran. The mixture was heated until 50° C. and filtered. The solution was cooled to −62° C., followed by addition of 54 mL of 2M NaO-tBu in tetrahydrofuran (108 mmol) in 2.5 h keeping the temperature between −55 and −60° C.

The temperature was allowed to increase to −20° C. and quenched with 200 mL of water. The mixture was transferred to another reactor using 30 mL of 2-methyltetrahydrofuran and the reaction mixture heated to 50° C. The pH was adjusted to 12 with 29 mL of 4N aqueous NaOH. The layers were separated. The organic phase was washed 2 times with 200 mL of a 5 w/w % aqueous NaCl solution, whereby the pH was adjusted to 12 using 4N aqueous NaOH, followed by 1 time with 100 mL of a 5 w/w % aqueous NaCl solution, whereby the pH was adjusted to 12 with 4N aqueous NaOH. Finally, the organic phase was washed with 100 mL of 5 w/w % aqueous NaHCO₃. The organic layer was evaporated to give a thick oil. The solid was re-crystallized from 200 mL of isopropanol to give 2-((4R,6S)-6-((E)-2-(2-cyclopropyl-4-(4-fluorophenyl)quinolin-3-yl)vinyl)-2,2-dimethyl-1,3-dioxan-4-yl)acetate, methyl ester as a white solid (32.1 g, yield 77.6%) with an HPLC purity of 99.7%.

The ¹H NMR data of this compound were in agreement with the literature data (WO 95/11898, Example 4).

### Example 3

### Preparation of 2-((4R,6S)-6-((E)-2-(2-cyclopropyl-4-(4-fluorophenyl)quinolin-3-yl)vinyl)-2,2-dimethyl-1,3-dioxan-4-yl)acetate, methyl ester from 2-cyclopropyl-4-(4-fluorophenyl)quinolin-3-carbaldehyde and 2-((4R,6S)-6-((benzo[d]thiazol-2-ylsulfonyl)methyl)-2,2-dimethyl-1,3-dioxan-4-yl)acetate methyl ester

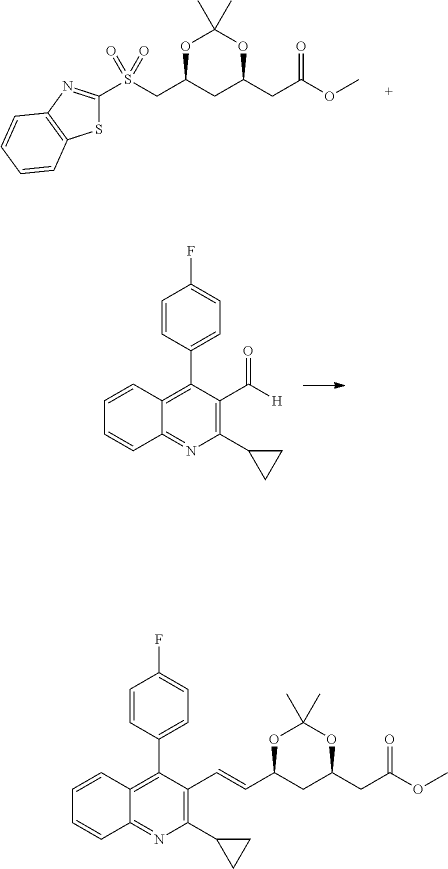 [SMILES: COC(=O)C[C@H]1C[C@@H](CS(=O)(=O)c2nc3ccccc3s2)OC(C)(C)O1 ; O=Cc1c(C2CC2)nc2ccccc2c1-c1ccc(F)cc1 ; COC(=O)C[C@H]1C[C@@H](/C=C/c2c(C3CC3)nc3ccccc3c2-c2ccc(F)cc2)OC(C)(C)O1]

2-((4R,6S)-6-((Benzo[d]thiazol-2-ylsulfonyl)methyl)-2,2-dimethyl-1,3-dioxan-4-yl)acetate methyl ester (30.0 g, 75 mmol) and 2-cyclopropyl-4-(4-fluorophenyl)quinolin-3-carbaldehyde (20.3 g, 70 mmol) were added to 35 mL of N-methyl-2-pyrrolidone and 200 mL of 2-methyltetrahydrofuran at 22° C. The reaction mixture was cooled to −60° C. Then 42 mL of 2M NaO-tBu in tetrahydrofuran (84 mmol) was added in 2.5 h keeping the temperature between −55 and −60° C. The temperature was allowed to increase to −50° C. and quenched with 100 mL of water. The mixture was transferred to another reactor using 30 mL of 2-methyltetrahydrofuran, heated to 50° C. and the pH adjusted to 12.6 with 31 mL of 4N aqueous NaOH. The layers were separated. The organic phase was washed 2 times with 100 mL of a 5 w/w % aqueous NaCl solution, whereby the pH was adjusted each time to 12 using 4N aqueous NaOH. Next, the organic phase was washed with 100 mL of 5 w/w % aqueous NaHCO₃. The organic layer was evaporated to give a thick oil. The solid was re-crystallized from 200 mL of isopropanol to give 2-((4R,6S)-6-((E)-2-(2-cyclopropyl-4-(4-fluorophenyl)quinolin-3-yl)vinyl)-2,2-dimethyl-1,3-dioxan-4-yl)acetate, methyl ester as a white solid (29.0 g, yield 81.3%) with an HPLC purity of 99.5%.

### Example 4

### Preparation of pitavastatin-Ca ((2a), R₄=Ca²⁺) from 2-((4R,6S)-6-((E)-2-(2-cyclopropyl-4-(4-fluorophenyl)quinolin-3-yl)vinyl)-2,2-dimethyl-1,3-dioxan-4-yl)acetate methyl ester ((1a), R₂=R₃=R₄=CH₃)

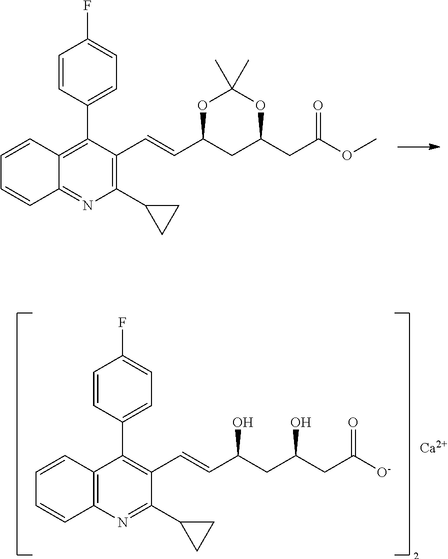

2-((4R,6S)-6-((E)-2-(2-cyclopropyl-4-(4-fluorophenyl)quinolin-3-yl)vinyl)-2,2-dimethyl-1,3-dioxan-4-yl)acetate methyl ester (10.0 g, 21.0 mmol) was added to acetonitrile (50 mL). The mixture was heated to 45° C., followed by addition of 4N aqueous HCl (5.3 mL, 21 mmol). The reaction mixture was stirred for 1.5 h and cooled to 22° C. Then in total 12 mL of 4N aqueous NaOH was added until pH 12.7. After stirring for 30 minutes, the pH was reduced to 9 by addition of acetic acid. The acetonitrile was removed via distillation under vacuum, followed by addition of 30 mL of water. To the clear solution was added over a period of 30 min, 47.3 mL of a solution of 4.5 w/w % Ca(OAc)₂.H₂O in water. Upon addition white precipitate was formed. After 1 h the precipitate was filtered, washed with water (2×15 mL) and dried to give 9.0 g of the calcium salt of pitavastatin as a solid. HPLC purity 98.8%, KF 2.1% water.

### Example 5

### Preparation of pitavastatin-Ca ((2a), R₄=Ca²⁺) from 2-((4R,6S)-6-((E)-2-(2-cyclopropyl-4-(4-fluorophenyl)quinolin-3-yl)vinyl)-2,2-dimethyl-1,3-dioxan-4-yl)acetate methyl ester ((1a), R₂=R₃=R₄=CH₃) via furfuryl amine salt

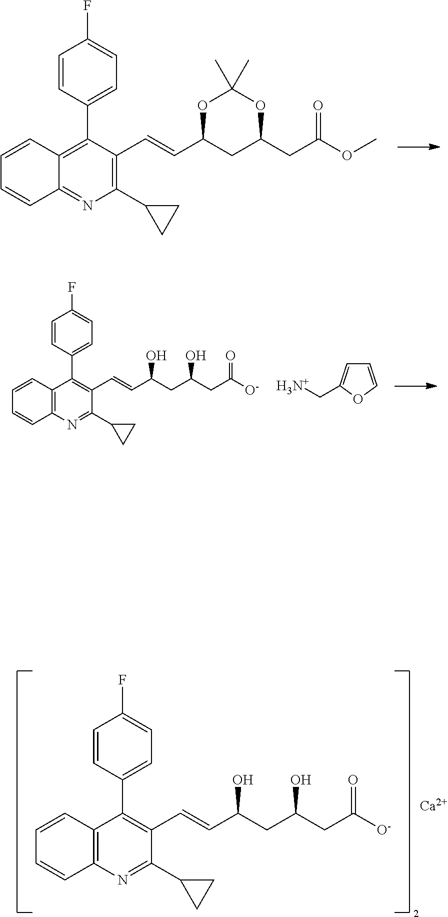

2-((4R,6S)-6-((E)-2-(2-cyclopropyl-4-(4-fluorophenyl)quinolin-3-yl)vinyl)-2,2-dimethyl-1,3-dioxan-4-yl)acetate methyl ester (10.0 g, 21.0 mmol) was added to acetonitrile (50 mL). The mixture was heated to 45° C. and 4N aqueous HCI (5 mL, 20 mmol) was added. The reaction was stirred for 2.5 h. After cooling to 22° C., 4M aqueous NaOH is added over a period of 1.5 h. The pH is reduced to 6.5 by addition of 1N aqueous HCl, and then concentrated under vacuum to remove the acetonitrile. Next methyl tert-butylether (20 mL) was added followed by addition of 1 N aqueous HCl until pH=4. The organic layer was separated and concentrated under vacuum. To the residue was added acetonitrile (68 mL) and water (3.9 mL). The reaction mixture was cooled to 10° C. after which furfurylamine (2.04 g, 21.0 mmol) dissolved in acetonitrile (13.5 mL) was added in 1.5 h. Upon addition, a white precipitate was formed. The reaction mixture was stirred for 30 min, followed by filtration of the pitavastatin-furfuryl amine salt. The salt was washed with ethyl acetate (2×10 mL) and dried to give 8.3 g of a white solid. The salt was added to water (100 mL) and the pH adjusted to 12.3 using 3.4 mL aqueous 4N NaOH. The reaction mixture is heated and 3×40 mL of water was removed via distillation under vacuum. After each distillation, the volume distilled water was replaced by adding the same volume of fresh water. After cooling to 22° C., 1 g of active carbon was added. The mixture was stirred for 1 h and the carbon removed by filtration. The pH of the solution was lowered by addition of acetic acid to 9.7 and 20 mL of water was added. Then over a period of 45 min, 33 mL of a solution of 4.5 w/w % Ca(OAc)₂.H₂O in water was added. Upon addition white precipitate was formed. After 30 minutes stirring, the solid was filtered and dried to give the calcium salt of Pitavastatin as a white solid (7.5 g, KF 2.8%). From the filtrate, the pitavastatin can be recovered in order to increase the overall yield. For example, this can be done, after acidification to pH=4 and extraction with methyl tert-butylether by formation of the amine salt as described in this example. In another embodiment, this filtrate can be combined with the extraction procedure as described in the example leading to a single step.

### Example 6

### Preparation of pitavastatin-acid from pitavastatin-Ca

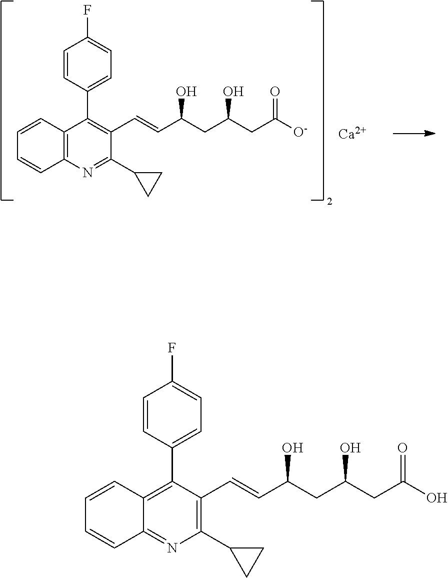

Pitavastatin-Ca (4.1 g) was suspended in water (30 mL) and ethyl acetate (30 mL) was added. The pH was adjusted to 4 using 0.5 N aqueous HCl. The organic layer was separated and concentrated under vacuum to a give foamy solid (3.7 g).

¹H NMR (300 MHz, DMSO-d6): δ=1.05-1.27 (m, 6H), 1.40-1.53 (m, 1H), 2.16-2.32 (m, 2H), 3.32-3.34 (bs, 2-OH, 2H), 3.72-3.82 (m, 1 H), 4.12-4.18 (m, 1 H), 5.61-5.68 (dd, J=5.7 Hz, J=16.1 Hz, 1H), 6.49-6.54 (dd, J=1.1 Hz, J=16.1 Hz, 1H), 7.26-7.42 (m, 6H), 7.62-7.68 (m, 1H) , 7.85-7.88 (d, J=8.4 Hz, 1H), 12.03 (bs, 1H).

The foamy solid was dissolved in acetonitrile (60 mL), divided into 3 portions of 20 mL and used as such in the next examples 7-9.

### Example 7

### Preparation of pitavastatin-tris(hydroxymethyl)aminomethane salt from pitavastatin acid

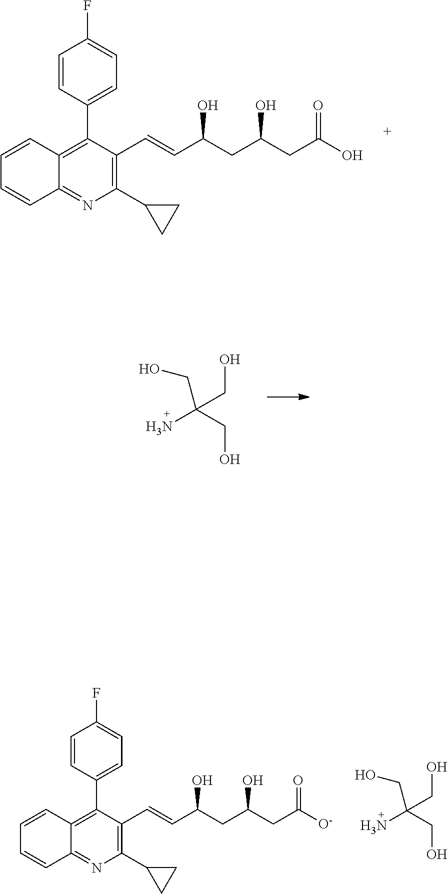 [SMILES: O=C(O)C[C@H](O)C[C@H](O)/C=C/c1c(C2CC2)nc2ccccc2c1-c1ccc(F)cc1 ; NC(CO)(CO)CO ; O=C([O-])C[C@H](O)C[C@H](O)/C=C/c1c(C2CC2)nc2ccccc2c1-c1ccc(F)cc1 ; NC(CO)(CO)CO]

To 20 mL of pitavastatin acid dissolved in acetonitrile (solution obtained as described in Example 6, was added (tris(hydroxymethyl) aminomethane (0.41 g, 3.4 mmol). The reaction mixture was heated until 50° C. to give complete dissolution. Then cooled in 1 h to 20° C. At 40° C., the salt precipitated. The slurry was stirred for 3 h at 20° C., followed by filtration of the pitavastatin-tris salt. The salt was washed with acetonitrile (2×5 mL) and dried to give 1.21 g pitavastatin-tris as a white solid.

¹H NMR (300 MHz, DMSO-d6): δ=1.03-1.24 (m, 6H), 1.35-1.48 (m, 1 H), 1.95-2.02 (dd, J=8.4 Hz, J=15.3 Hz, 1H), 2.09-2.16 (dd, J=4.3 Hz, J=15.3 Hz, 1H), 3.36 (s, 6H), 3.51-4.25 (very broad, 8H), 3.59-3.67 (m, 1H), 4.11-4.17 (m, 1H), 5.61-5.68 (dd, J=5.7 Hz, J=16.1 Hz, 1H), 6.47-6.52 (dd, J=1.1 Hz, J=16.1 Hz, 1H), 7.25-7.41 (m, 6H), 7.61-7.67 (m, 1 H) , 7.85-7.88 (d, J=8.4 Hz, 1 H).

### Example 8

### Preparation of pitavastatin-lysine salt from pitavastatin acid

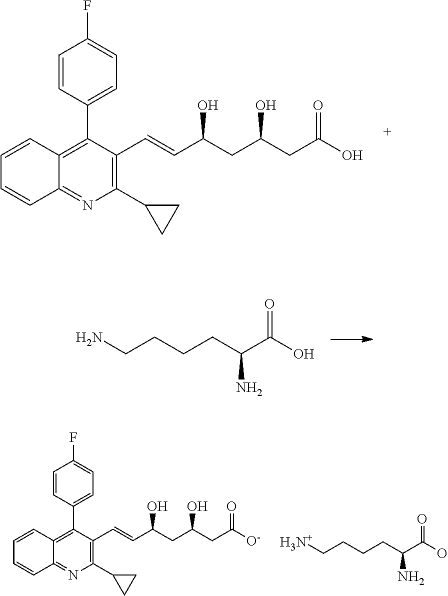 [SMILES: O=C(O)C[C@H](O)C[C@H](O)/C=C/c1c(C2CC2)nc2ccccc2c1-c1ccc(F)cc1 ; NCCCC[C@H](N)C(=O)O ; O=C([O-])C[C@H](O)C[C@H](O)/C=C/c1c(C2CC2)nc2ccccc2c1-c1ccc(F)cc1 ; N[C@@H](CCCC[NH3+])C(=O)O]

To 20 mL of pitavastatin acid dissolved in acetonitrile (solution obtained as described in Example 6, was added L-lysine (0.50 g, 3.4 mmol) dissolved in 2 mL of water. Next, methanol was added (5 mL). The reaction mixture was heated until 40° C. and cooled in 30 min to 20° C. to give a very thick slurry. The slurry was heated again to 40° C. and cooled to 20° C. in 1 h. The slurry was stirred for 4 h at 20° C., followed by filtration of the pitavastatin-lysine salt. The salt was washed with acetonitrile (2×2.5 mL) and dried to give 1.39 g pitavastatin-lysine salt as a slightly yellow solid.

¹H NMR (300 MHz, DMSO-d6): δ=1.02-1.23 (m, 6H), 1.35-1.48 (m, 1 H), 1.37-1.54 (m, 4H), 1.60-1.72 (m, 2H), 1.91-1.99 (m, 1H), 2.09-2.16 (m, 1H), 2.70-2.74 (m, 2H), 3.18-3.22 (m, 1H), 3.58-3.65 (m, 1H), 3.81-4.50 (very broad, 7H), 4.11-4.17 (m, 1H), 5.58-5.67 (dd, J=5.7 Hz, J=16.1 Hz, 1H), 6.47-6.52 (dd, J=1.1 Hz, J=16.1 Hz, 1H), 7.25-7.42 (m, 6H), 7.61-7.67 (m, 1H) , 7.84-7.87 (d, J=8.1 Hz, 1H), COOH not seen in DMSO-d6.

### Example 9

### Preparation of pitavastatin-choline salt from pitavastatin acid

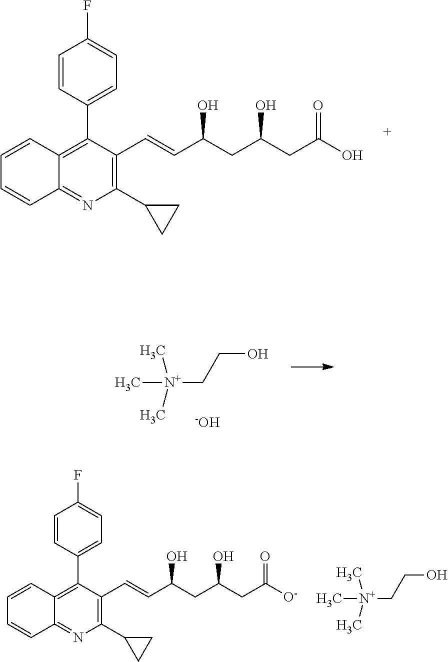

To 20 mL of the pitavastatin acid dissolved in acetonitrile (obtained as described in Example 6, was added choline (0.41 g, 3.9 mmol). Choline (2-hydroxyethyl)trimethylammonium) was added as a 45 w/w % solution in methanol as the OH salt. The acetonitrile was removed by distillation and fresh acetonitrile (25 mL) was added, whereupon precipitation was observed. The slurry was stirred for 3 h at 20° C., followed by filtration of the pitavastatin-choline salt. The salt was washed with acetonitrile (2×2.5 mL) and dried to give 0.95 g pitavastatin-choline salt as a white solid.

¹H NMR (300 MHz, DMSO-d6): δ=1.02-1.23 (m, 4H), 1.19-1.24 (m, 2H), 1.32-1.42 (m, 1H), 1.70-1.77 (m, 1H), 1.92-1.99 (m, 1H), 3.11-3.17 (s, 9H), 3.36-3.45 (m, 6H), 3.83-3.88 (m, 2H), 4.11-4.17 (m, 1H), 5.57-5.64 (dd, J=5.7 Hz, J=16.1 Hz, 1H), 6.46-6.53 (dd, J=1.1 Hz, J=16.1 Hz, 1H), 7.26-7.42 (m, 6H), 7.61-7.66 (m, 1H), 7.85-7.88 (d, J=8.1 Hz, 1H).

### Example 10

### Solubility of pharmaceutically acceptable salts of pitavastatin

The pharmaceutically acceptable pitavastatin salts obtained above were subjected to solubility measurements. In addition the calcium salt of pitavastatin and the 1-furfurylamine salt of pitavastatin were included (a basic toxicological evaluation has indicated that also 1-furfurylamine can be classified as very safe). Hence, the respective salts were contacted with buffers of pH 1.2 and pH 4.8 respectively. Amounts of added salt were increased as long as the resulting solution remained clear and the resulting pH value, as well as the total amount of salt added, are reported in the below Table.

| | | | Pitavastatin | Pitavastatin salt |
|---|---|---|---|---|
| | | | mg · g⁻¹ | mg · g⁻¹ |
| | | | solution | solution |
| Pitavastatin salt | pH | Appearance | (calculated) | (actual) |
| Calcium salt | 5.7 | Suspension | 0 | 0 |
| Calcium salt | 6.8 | Suspension | 2 | 2 |
| Furfurylamine salt | 5.8 | Suspension | 1 | 1 |
| Furfurylamine salt | 6.8 | Suspension | 65 | 81 |
| L-Lysine salt | 6.1 | Suspension | 48 | 64 |
| L-Lysine salt | 4.5 | Suspension | 0 | 0 |
| L-Lysine salt | 8.0 | Clear | 163 | 221 |
| Choline salt | 6.9 | Clear | 169 | 211 |
| Choline salt | 7.3 | Clear | 162 | 202 |
| Tris(hydroxymethyl)- | 8.0 | Clear | 163 | 222 |
| aminomethane salt | | | | |
| Tris(hydroxymethyl)- | 8.3 | Clear | 161 | 219 |
| aminomethane salt | | | | |

The following observations were made. The solubility of pitavastatin calcium salt is very low, initially the compound floated on the buffer. In contrast, the solubility of the amine salts was much higher; in many cases solutions were still clear after more than 160 mg·g⁻¹ solution was added. For the amine salts, pH values increased to values as reported in the Table upon increasing addition of salt (indicating exhaustion of buffer capacity due to high salt solubility). In case of the L-lysine salt of pitavastatin, the entry at pH 6.1 was obtained starting with a buffer of pH 1.2 whereas the entry at pH 4.5 was obtained starting with a buffer of pH 4.8 where the solubility was very low; this seems to suggest that the presence of amine increases the solubility of pitavastatin. Qualitatively, the speed of dissolution of the amine salts was much faster than the speed of dissolution of the calcium salt. The pitavastatin amine salts were not hygroscopic (qualitative determination).

1. A pharmaceutically acceptable amine salt of pitavastatin, wherein said amine is selected from the group consisting of aminopolyols and tetraalkyl ammonium salts.

2. The pharmaceutically acceptable amine salt of pitavastatin of claim 1 wherein said aminopolyol is tromethamine.

3. The pharmaceutically acceptable amine salt of pitavastatin of claim 1 wherein said tetraalkyl ammonium salt is choline.

4. A method for the preparation of an amine salt of pitavastatin comprising reacting pitavastatin acid or pitavastatin calcium salt with an amine in a solvent followed by precipitating said amine salt of pitavastatin, wherein said reacting is carried out at a first temperature and said precipitating is carried out at a second temperature that is at least 5° C. below said first temperature.

5. Method according to claim 4 comprising the steps of:
a) Contacting a protected derivative of pitavastatin with acid followed by base or with base followed by acid;
b) Treating the mixture obtained in step a) with an amine;
Isolating the amine salt obtained in step b).

6. Method according to claim 4 wherein said amine is selected from the group consisting of amino acids, aminopolyols, amino sugars, ammonia, ethyl amine derivatives, guanines, purines, tetraalkyl ammonium salts and vitamins.

7. Method according to claim 6 wherein said amine is an amino acid selected from the group consisting of histidine, lysine and ornithine.

8. Method according to claim 6 wherein said amine is tromethamine.

9. Method according to claim 6 wherein said amine is an amino sugar selected from the group consisting of daunosamine, galactosamine, glucosamine and N-methylglucamine.

10. Method according to claim 6 wherein said amine is an ethyl amine derivative selected from the group consisting of benzathine, diethyl amine, ethanol amine, ethyl amine, ethylene diamine, 1-(2-hydroxyethyl)-pyrrolidine, piperazine, triethanol amine and triethyl amine.

11. Method according to claim 6 wherein said amine is a tetraalkyl ammonium salt selected from the group consisting of carnitine and esters thereof, choline, tetraethyl ammonium and tetramethyl ammonium.

12. Method according to claim 6 wherein said amine is a vitamin selected from the group consisting of vitamin B1, vitamin B3, vitamin B6 and vitamin B11.

13. A pharmaceutical composition comprising the amine salt of claim 1 or a pharmaceutically acceptable hydrate or solvate thereof and one or more pharmaceutically acceptable carriers or excipients.
